(19) United States
(12) Patent Application Publication
(10) Pub. No.: US 2023/0091196 A1
(43) Pub. Date: Mar. 23, 2023

(54) MEDICAL IMAGE PROCESSING APPARATUS, MEDICAL IMAGE PROCESSING METHOD, AND COMPUTER PROGRAM PRODUCT

(71) Applicant: CANON MEDICAL SYSTEMS CORPORATION, Otawara-shi (JP)

(72) Inventor: Shigeharu OHYU, Yaita (JP)

(73) Assignee: CANON MEDICAL SYSTEMS CORPORATION, Otawara-shi (JP)

(21) Appl. No.: 17/948,456

(22) Filed: Sep. 20, 2022

(30) Foreign Application Priority Data
Sep. 22, 2021 (JP) 2021-154485

(51) Int. Cl. G06T 5/00; G06T 7/20; G06T 11/00; G06T 7/90; G09G 3/20; A61B 6/00

(52) U.S. Cl. CPC G06T 5/009; G06T 7/20; G06T 11/001; G06T 7/90; G09G 3/20; A61B 6/481; A61B 6/5217; A61B 6/463; G06T 2207/10024; G06T 2210/41; G06T 2207/10081; G06T 2207/20208; G06T 2207/20212; G09G 2320/066; G09G 2320/0242

(57) ABSTRACT

A medical image processing apparatus according to an embodiment includes processing circuitry. The processing circuitry is configured to obtain contrast-enhanced images related to an examined subject and corresponding to a plurality of temporal phases. On the basis of data values of pixels in the contrast-enhanced images corresponding to the plurality of temporal phases, the processing circuitry is configured to determine which one of contrast enhancement dominance and fluid accumulation dominance corresponds to the pixels or a region including the pixels. The processing circuitry is configured to generate a display mode based on the determination.

### CROSS-REFERENCE TO RELATED APPLICATIONS

This application is based upon and claims the benefit of priority from Japanese Patent Application No. 2021-154485, filed on Sep. 22, 2021; the entire contents of which are incorporated herein by reference.

### FIELD

Embodiments described herein relate generally to a medical image processing apparatus, a medical image processing method, and a computer program product.

### BACKGROUND

Conventionally, in medical image diagnosing processes using an X-ray Computed Tomography (CT) apparatus or a Magnetic Resonance Imaging (MRI) apparatus, contrast-enhanced dynamic examination may be performed in some situations. The contrast-enhanced dynamic examination determines, for example, the presence/absence of hemorrhage into the abdominal cavity and damage to arteries, veins, or the portal vein, on the basis of a plurality of images of which the contrast is enhanced in a plurality of temporal phases.

However, when the presence/absence of hemorrhage or damage is determined while visually comparing, with one another, the plurality of images of which the contrast is enhanced in the plurality of temporal phases, a problem arises where the medical image diagnosing process requires a long period of time.

### BRIEF DESCRIPTION OF THE DRAWINGS

FIG. 1 is a diagram illustrating an exemplary configuration of an X-ray Computed Tomography (CT) apparatus having installed therein a medical image processing apparatus according to an embodiment;

FIG. 2 is a chart for explaining a display mode generating process in a medical image processing process according to the embodiment;

FIG. 3 is a flowchart illustrating an example of the medical image processing process according to the embodiment;

FIG. 4 is a drawing illustrating an example of display modes generated in the medical image processing process according to the embodiment; and

FIG. 5 is a drawing illustrating another example of the display modes generated in the medical image processing process according to the embodiment.

### DETAILED DESCRIPTION

A medical image processing apparatus according to an embodiment includes processing circuitry. The processing circuitry is configured to obtain contrast-enhanced images related to an examined subject and corresponding to a plurality of temporal phases. On the basis of data values of pixels in the contrast-enhanced images corresponding to the plurality of temporal phases, the processing circuitry is configured to determine which one of contrast enhancement dominance and fluid accumulation dominance corresponds to the pixels or a region including the pixels. The processing circuitry is configured to generate a display mode based on the determination.

Exemplary embodiments of a medical image processing apparatus, an X-ray CT apparatus, a medical image processing method, a program, and a computer program product will be explained below, with reference to the accompanying drawings. In the following description, some of the constituent elements having functions that are the same as or similar to those already described with reference to previously-explained drawings will be referred to by using the same reference characters, and duplicate explanations will be provided only when necessary. Further, some drawings may illustrate mutually the same parts in mutually-different dimensions or proportions. Further, from the viewpoint of maintaining recognizability of the drawings, for example, reference characters may be used only for relevant or representative constituent elements in the description of the drawings. Some of the constituent elements, including those having the same or similar functions, may not have reference characters.

In the embodiments described below, examples will be explained in which a medical image processing apparatus according to each of the embodiments is installed in an X-ray Computed Tomography (CT) apparatus.

The medical image processing apparatus according to each of the embodiments does not necessarily have to be installed in an X-ray CT apparatus and may instead be realized as an independent apparatus, by using a computer that includes, as hardware resources thereof, a processor such as a Central Processing Unit (CPU) and memory elements such as a Read-Only Memory (ROM) and a Random Access Memory (RAM). In that situation, the processor installed in the computer is able to realize various types of functions according to the embodiments, by executing a program that is read from the ROM or the like and loaded into the RAM.

Further, the medical image processing apparatus according to any of the embodiments may be realized as being installed in a medical image diagnosis apparatus other than the X-ray CT apparatus. In that situation, a processor installed in each medical image diagnosis apparatuses is able to realize the functions according to any of the embodiments by executing a program read from a ROM or the like and loaded into a RAM. Examples of the other medical image diagnosis apparatus include various types of medical image diagnosis apparatuses such as a Magnetic Resonance Imaging (MRI) apparatus, a Single Photon Emission Computed Tomography (SPECT)-CT apparatus in which a SPECT apparatus and an X-ray CT apparatus are integrally formed, and a Positron Emission computed Tomography (PET)-CT apparatus in which a PET apparatus and an X-ray CT apparatus are integrally formed. Further, an X-ray diagnosis apparatus such as an X-ray rotation angiography apparatus may be used as the other medical image diagnosis apparatus. In other words, for example, cone beam CT images reconstructed on the basis of a plurality of projection X-ray images acquired by an X-ray diagnosis apparatus while rotating a C-arm may also be used as at least a part of the images related to an examined subject and corresponding to a plurality of temporal phases, similarly to CT images in any of the embodiments.

For example, there are various types of X-ray CT apparatuses such as third-generation CT and fourth-generation CT apparatuses. It is possible to apply any of these various types to the embodiments. In this situation, the third-generation CT apparatuses are of a Rotate/Rotate type in which an X-ray tube and a detector integrally rotate around an examined subject. The fourth-generation CT apparatuses are of a Stationary/Rotate type in which only an X-ray tube rotates around an examined subject while a large number of X-ray detecting elements arrayed in a ring formation are fixed.

### First Embodiment

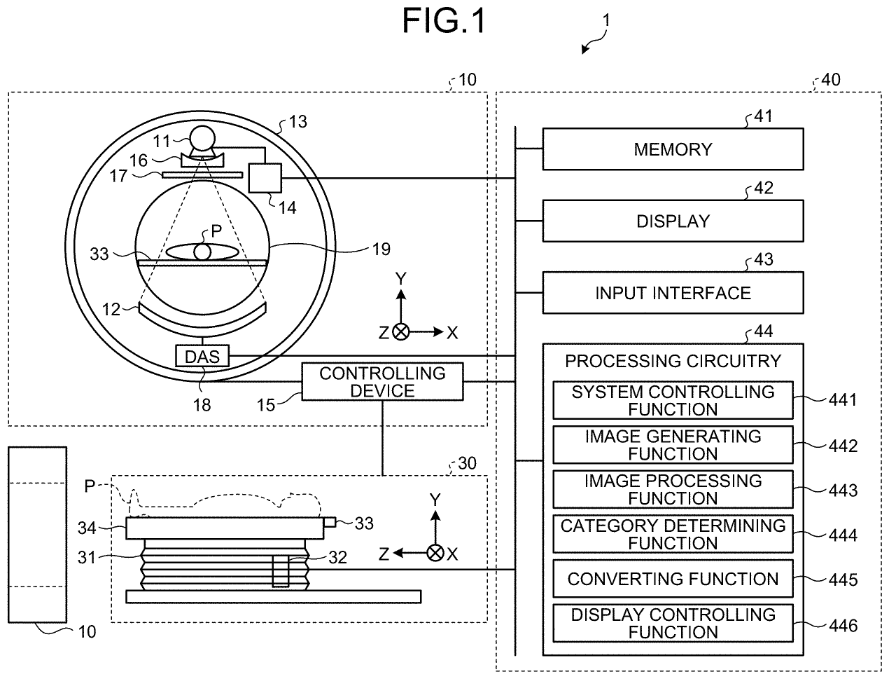

FIG. 1 is a diagram illustrating an exemplary configuration of an X-ray CT apparatus 1 having installed therein a medical image processing apparatus according to an embodiment. The X-ray CT apparatus 1 is configured to emit X-rays onto an examined subject (hereinafter, “patient”) P from an X-ray tube 11, so that an X-ray detector 12 detects the emitted X-rays. The X-ray CT apparatus 1 is configured to generate a CT image related to the patient P, on the basis of an output from the X-ray detector 12.

As illustrated in FIG. 1, the X-ray CT apparatus 1 includes a gantry 10, a table 30, and a console 40. For the sake of convenience in the explanation, the gantry 10 is depicted more than once in FIG. 1. The gantry 10 is a scan device including a configuration for performing X-ray CT imaging on the patient P. The table 30 is a transporting device on which the patient P undergoing the X-ray CT imaging is placed, so as to determine the position of the patient P. The console 40 is a computer configured to control the gantry 10. For example, the gantry 10 and the table 30 may be provided in a CT examination room, while the console 40 may be provided in a control room adjacent to the CT examination room. The gantry 10, the table 30, and the console 40 are connected so as to be able to communicate with one another in a wired or wireless manner.

In this situation, the console 40 does not necessarily have to be provided in the control room. For example, the console 40 may be provided in the same room as the gantry 10 and the table 30 are. Alternatively, the console 40 may be incorporated in the gantry 10.

Further, in the present embodiment, the rotation axis of a rotating frame 13 in a non-tilt state or the long-axis directions of a tabletop 33 of the table 30 is defined as a Z-axis direction; an axial direction orthogonal to the Z-axis direction and parallel to the floor surface is defined as an X-axis direction; and an axial direction orthogonal to the Z-axis direction and perpendicular to the floor surface is defined as a Y-axis direction.

As illustrated in FIG. 1, the gantry 10 includes the X-ray tube 11, the X-ray detector 12, the rotating frame 13, an X-ray high-voltage device 14, a controlling device 15, a wedge 16, a collimator 17, and a data acquisition circuit (Data Acquisition System, DAS) 18.

The X-ray tube 11 is a vacuum tube having a negative pole (a filament) that generates thermo electrons and a positive pole (a target) that generates X-rays in response to collisions of the thermos electrons. The X-ray tube 11 is configured to emit the X-rays onto the patient P, by emitting the thermo electrons from the negative pole toward the positive pole, while using high voltage supplied from the X-ray high-voltage device 14.

In this situation, the hardware used for generating the X-rays is not limited to the X-ray tube 11. For example, it is acceptable to use a fifth generation scheme for generating the X-rays, in place of the X-ray tube 11. The fifth generation scheme includes a focus coil configured to converge an electron beam generated from an electron gun; a deflection coil that causes an electromagnetic deflection; and a target ring that covers a half of the surrounding of the patient P and is configured to generate X-rays as a result of having the deflected electron beam collide therewith.

The X-ray detector 12 is configured to detect X-rays that were emitted from the X-ray tube 11 and have passed through the patient P and is configured to output an electrical signal corresponding to a radiation amount of the detected X-rays to the DAS 18. For example, the X-ray detector 12 includes an X-ray detecting element row in which a plurality of X-ray detecting elements are arranged in a channel direction along an arc while being centered on the focal point of the X-ray tube 11. For example, the X-ray detector 12 has a structure in which a plurality of sets each including X-ray detecting elements in the channel direction are arranged in a slice direction (a row direction). Further, for example, the X-ray detector 12 may be a detector of an indirect conversion type having a grid, a scintillator array, and an optical sensor array. The scintillator array has a plurality of scintillators. Each of the scintillators has a scintillator crystal that outputs light having a photon quantity corresponding to the amount of incident X-rays. The grid is arranged on a surface of the scintillator array that is positioned on the X-ray incident side and includes an X-ray blocking plate having a function of absorbing scattered X-rays. The grid may be referred to as a collimator (a one-dimensional collimator or a two-dimensional collimator) in some situations. The optical sensor array has a function of converting the photon quantities of the light from the scintillators into corresponding electrical signals. As optical sensors, Photomultiplier Tubes (PMTs) may be used, for example. Alternatively, the X-ray detector 12 may be a detector of a direct conversion type that includes a semiconductor element configured to convert incident X-rays into an electrical signal. In this situation, the X-ray detector 12 is an example of a detecting unit.

The rotating frame 13 is an annular frame configured to support the X-ray tube 11 and the X-ray detector 12 so as to oppose each other and configured to rotate the X-ray tube 11 and the X-ray detector 12 via the controlling device 15 (explained later). An Field Of View (FOV) is set with an opening part 19 of the rotating frame 13. For example, the rotating frame 13 is cast by using aluminum or the like. In addition to the X-ray tube 11 and the X-ray detector 12, the rotating frame 13 is also capable of further supporting the X-ray high-voltage device 14, the wedge 16, the collimator 17, the DAS 18, and the like. Furthermore, the rotating frame 13 is also capable of further supporting various types of elements that are not illustrated in FIG. 1.

The X-ray high-voltage device 14 includes a high-voltage generating device and an X-ray controlling device. The high-voltage generating device includes electrical circuits such as a transformer, a rectifier, and the like and is configured to generate the high voltage to be applied to the X-ray tube 11 and a filament current to be supplied to the X-ray tube 11. The X-ray controlling device is configured to control output voltage corresponding to the X-rays to be emitted by the X-ray tube 11. The high-voltage generating device may be of a transformer type or an inverter type. Further, the X-ray high-voltage device 14 may be provided on the rotating frame 13 in the gantry 10 or may be provided on a fixed frame (not illustrated) in the gantry 10. In this situation, the fixed frame is a frame configured to rotatably support the rotating frame 13.

The controlling device 15 includes a driving mechanism such as a motor and an actuator and processing circuitry including a processor configured to control the driving mechanism and a memory or the like. The controlling device 15 is configured to control operations of the gantry 10 and the table 30, upon receipt of input signals from an input interface 43 and an input interface provided for the gantry 10, or the like. Examples of the operation control exercised by the controlling device 15 include: control to rotate the rotating frame 13, control to tilt the gantry 10, and control to bring the table 30 into operation. Further, the control to tilt the gantry 10 may be realized as a result of the controlling device 15 turning the rotating frame 13 on an axis extending parallel to the X-axis direction, according to inclination angle (tilt angle) information input through an input interface attached to the gantry 10. Further, the controlling device 15 may be provided for the gantry 10 or for the console 40.

The wedge 16 is a filter used for adjusting the X-ray amount of the X-rays emitted from the X-ray tube 11. More specifically, the wedge 16 is a filter configured to pass and attenuate the X-rays emitted from the X-ray tube 11, so that the X-rays emitted from the X-ray tube 11 onto the patient P has a predetermined distribution. The wedge 16 may be a wedge filter or a bow-tie filter, for example, and is obtained by processing aluminum or the like so as to have a predetermined target angle and a predetermined thickness.

The collimator 17 is configured to limit an emission range of the X-rays that have passed through the wedge 16. The collimator 17 is configured to slidably support a plurality of lead plates blocking X-rays and is configured to adjust the forms of slits shaped by the plurality of lead plates. Further, the collimator 17 may be referred to as an X-ray limiter.

The DAS 18 is configured to read, from the X-ray detector 12, the electrical signal corresponding to the radiation amount of the X-rays detected by the X-ray detector 12. The DAS 18 is configured to acquire detection data having a digital value corresponding to the radiation amount of the X-rays during a view period, by amplifying the read electrical signal and integrating (adding up) the electrical signals over the view period. The detection data may be referred to as projection data. For example, the DAS 18 is realized by using an Application Specific Integrated Circuit (ASIC) including a circuit element capable of generating the projection data. The projection data is transferred to the console 40 via a contactless data transfer device or the like. In this situation, the DAS 18 is an example of a detecting unit.

Further, the detection data generated by the DAS 18 is transmitted, via optical transmission, from a transmitter provided on the rotating frame 13 and including a Light Emitting Diode (LED), to a receiver provided in a non-rotating part (e.g., the fixed frame; not illustrated in FIG. 1) of the gantry 10 and including a photodiode, so as to be further transferred to the console 40. In this situation, possible methods for transmitting the detection data from the rotating frame 13, which is a rotating part, to the non-rotating part of the gantry 10 are not limited to the optical communication described above, and it is acceptable to adopt any contactless data transfer method.

In the present embodiment, an example is explained in which the X-ray CT apparatus 1 has installed therein the integral-type X-ray detector 12; however, the techniques of the present embodiment may also be realized with an X-ray CT apparatus 1 having installed therein a photon-counting X-ray detector.

The table 30 is a device on which the patient P to be scanned is placed and moved. The table 30 includes a base 31, a table driving device 32, the tabletop 33, and a supporting frame 34. The base 31 is a casing configured to movably support the supporting frame 34 in vertical directions. The table driving device 32 is a driving mechanism configured to move the tabletop 33 on which the patient P is placed, in the long-axis directions of the tabletop 33. The table driving device 32 includes a motor and an actuator or the like. The tabletop 33 is a board on which the patient P is placed. The tabletop 33 is provided on the top face of the supporting frame 34. The tabletop 33 is capable of projecting from the table 30 toward the gantry 10, to make it possible to image the whole body of the patient P. The tabletop 33 may be formed, for example, by using Carbon Fiber Reinforced Plastic (CFRP), which has excellent X-ray transmissibility and physical properties such as rigidity and strength. Further, for example, the tabletop 33 may be hollow inside. The supporting frame 34 is configured to support the tabletop 33 so as to be movable in the long-axis directions of the tabletop 33. In addition to moving the tabletop 33, the table driving device 32 may also be configured to move the supporting frame 34 in the long-axis directions of the tabletop 33.

The console 40 includes a memory 41, a display 42, the input interface 43, and processing circuitry 44. Data communication among the memory 41, the display 42, the input interface 43, and the processing circuitry 44 is performed via a bus. Further, although the example is explained in which the console 40 and the gantry 10 are separate from each other, the gantry 10 may include the console 40 or a part of the constituent elements of the console 40.

The memory 41 is realized by using, for example, a semiconductor memory element such as a ROM, a RAM, or a flash memory, or a hard disk, an optical disk, or the like. For example, the memory 41 is configured to store therein the projection data and reconstructed image data. Further, for example, the memory 41 is configured to store therein various types of programs. In this situation, a storage area of the memory 41 may be provided inside the X-ray CT apparatus 1 or may be provided in an external storage device connected via a network. In this situation, the memory 41 is an example of a storage unit.

The display 42 is configured to display various types of information. For example, the display 42 is configured to display a medical image (a CT image) generated by the processing circuitry 44, a Graphical User Interface (GUI) used for receiving various types of operations from an operator, and the like. Information displayed on the display 42 includes various types of display modes generated by the medical image processing apparatus according to the embodiment. In an example, the display 42 is configured to display one selected from between a color image and a color superimposed image in which the color image is superimposed, the color image being obtained by determining one or both of a hue or brightness of each of the pixels on the basis of a result of a category determination according to the embodiment. As the display 42, it is possible to use any of various types of arbitrary display devices, as appropriate. For example, as the display 42, it is possible to use a Liquid Crystal Display (LCD) device, a Cathode Ray Tube (CRT) display device, an Organic Electroluminescence Display (OELD) device, or a plasma display device.

In this situation, the display 42 may be provided in any location in the control room. Further, the display 42 may be provided for the gantry 10. Also, the display 42 may be of a desktop type or may be configured by using a tablet terminal or the like capable of wirelessly communicating with the main body of the console 40. Alternatively, one or more projectors may be used as the display 42. In these situations, the display 42 is an example of a display unit.

The input interface 43 is configured to receive various types of input operations from the operator, to convert the received input operations into electrical signals, and to output the electrical signals to the processing circuitry 44. For example, the input interface 43 is configured to receive, from the operator, an acquisition condition used at the time of acquiring the projection data, a reconstruction condition used at the time of reconstructing the CT image, an image processing condition used at the time of generating a post-processing image from the CT image, and the like. Further, for example, the input interface 43 is configured to receive, from the operator, operations related to setting and changing the various types of display modes generated by the medical image processing apparatus according to the embodiment.

As the input interface 43, it is possible to use, for example, a mouse, a keyboard, a trackball, a switch, a button, a joystick, a touchpad, and a touch panel display device, and/or the like, as appropriate. Further, in the present embodiment, the input interface 43 does not necessarily have to include those physical operation components. For instance, possible examples of the input interface 43 include electrical signal processing circuitry configured to receive an electrical signal corresponding to an input operation from an external input device provided separately from the apparatus and to output the electrical signal to the processing circuitry 44. Also, the input interface 43 may be provided for the gantry 10. Further, the input interface 43 may be configured by using a tablet terminal or the like capable of wirelessly communicating with the main body of the console 40. In this situation, the input interface 43 is an example of an input unit.

The processing circuitry 44 is configured to control operations of the entirety of the X-ray CT apparatus 1. As hardware resources thereof, the processing circuitry 44 includes a processor and memory elements such as a ROM, a RAM, and/or the like. By employing the processor configured to execute programs loaded into a memory, the processing circuitry 44 is configured to implement a system controlling function 441, an image generating function 442, an image processing function 443, a category determining function 444, a converting function 445, a display controlling function 446, and the like. In this situation, the processing circuitry 44 is an example of a processing unit.

By employing the system controlling function 441, the processing circuitry 44 is configured to control the various types of functions of the processing circuitry 44, on the basis of the input operations received from the operator via the input interface 43. For example, the system controlling function 441 is configured to control a CT scan performed by the gantry 10. The system controlling function 441 is configured to obtain the detection data acquired in the CT scan. The detection data acquired in the CT scan includes a plurality of pieces of detection data related to a plurality of temporal phases. In an example, the system controlling function 441 may obtain the detection data related to the patient P from the outside of the X-ray CT apparatus 1. Further, for the display mode generating process according to the embodiment, the system controlling function 441 is configured to obtain CT images related to the patient and corresponding to a plurality of temporal phases, from the memory 41, for example. In this situation, the processing circuitry 44 realizing the system controlling function 441 is an example of an obtaining unit.

By employing the image generating function 442, the processing circuitry 44 is configured to generate data obtained by performing, on the detection data output from the DAS 18, pre-processing processes such as a logarithmic conversion process, an offset correction process, an inter-channel sensitivity correction process, a beam hardening correction, and/or the like. The image generating function 442 is configured to store the generated data into the memory 41. Further, the data before the pre-processing processes (the detection data) and the data after the pre-processing processes may collectively be referred to as projection data. The image generating function 442 is configured to generate CT image data by performing a reconstruction process that implements a filter backprojection method, a successive approximation reconstruction method, machine learning, or the like, on the generated projection data (i.e., the projection data after the pre-processing processes). The image generating function 442 is configured to store the generated CT image data into the memory 41.

By employing the image processing function 443, the processing circuitry 44 is configured to convert the CT image data generated by the image generating function 442 into tomographic image data on an arbitrary cross-sectional plane or three-dimensional image data by using a publicly-known method, on the basis of an input operation received from the operator via the input interface 43. For example, the image processing function 443 is configured to generate rendering image data in an arbitrary viewpoint direction by performing, on the CT image data, a three-dimensional image processing process such as volume rendering, surface rendering, an image value projection process, a Multi-Planar Reconstruction (MPR) process, or a Curved MPR (CPR) process. Alternatively, three-dimensional image data (i.e., volume data) such as the rendering image data in the arbitrary viewpoint direction may directly be generated by the image generating function 442.

As explained above, the system controlling function 441, the image generating function 442, and the image processing function 443 are configured to generate the image data related to the patient P and corresponding to the plurality of temporal phases. In the present embodiment, an example will be explained in which a plain CT image, a pre-contrast enhancement CT image, an artery dominant phase CT image, and a parenchymal phase CT image are used as examples of the images related to the patient P and taken at mutually-different points in time. In other words, in the present embodiment, the image data related to the patient P and corresponding to the plurality of temporal phases may include, for example, plain CT image data acquired prior to the start of contrast enhancement i.e., by plain CT, CT image data in an artery dominant phase acquired by contrast-enhanced CT, and CT image data in a parenchymal phase acquired by contrast-enhanced CT. Further, in the present embodiment, when the pieces of image data related to the patient P and corresponding to the plurality of temporal phases are not distinguished from one another, the image data may simply be referred to as dynamic CT image data.

Further, the image processing function 443 is configured to select a target region for which a color image is to be generated, from the CT image of an entire range read by the system controlling function 441 from the memory 41. Further, the image processing function 443 is configured to perform motion compensation and a filtering process on the contrast-enhanced images corresponding to the mutually-different temporal phases. Also, the image processing function 443 is configured to generate a contrast enhancement intensity map, a contrast enhancement change map, a local uniformity map, a pre-contrast enhancement CT value map, and a mask image. Further, the image processing function 443 is configured to generate, as a base image, a maximum value image among the different temporal phases, as compared among the plain CT image, the artery dominant phase CT image, and the parenchymal phase CT image that were motion-compensated. In addition, the image processing function 443 is configured to generate a color superimposed image by adding a color image to the base image. The image processing function 443 is configured to store the generated various types of image data into the memory 41. In this situation, the processing circuitry 44 realizing the image processing function 443 is an example of an image processing unit.

By employing the category determining function 444, the processing circuitry 44 is configured to normalize the pixel values in the contrast enhancement intensity map, the contrast enhancement change map, the local uniformity map, the pre-contrast enhancement CT value map, and the mask image. Further, on the basis of at least one of the pixel values in the contrast enhancement intensity map, the contrast enhancement change map, the local uniformity map, the pre-contrast enhancement CT value map, and the mask image, the category determining function 444 is configured to determine which one of the following two categories each of the pixels belongs to. Examples of the two categories include categories such as “local uniformity dominance” and “contrast enhancement dominance”. As explained herein, the category determining function 444 is configured to determine which one of contrast enhancement dominance and fluid accumulation dominance corresponds to each of the pixels, on the basis of the data values of the pixels in the CT images corresponding to the plurality of temporal phases. In this situation, the processing circuitry 44 realizing the category determining function 444 is an example of a determining unit. Further, the categories such as the local uniformity dominance and the contrast enhancement dominance are examples of the first state and the second state.

By employing the converting function 445, the processing circuitry 44 is configured to generate display modes based on the determination by the category determining function 444. More specifically, the converting function 445 is configured to determine a hue and a brightness level of each of the pixels, by using color conversion mathematical functions each of which is associated with a different one the categories determined by the category determining function 444. Further, in accordance with determined color codes, the converting function 445 is configured to generate a color image in which each of the pixels has values corresponding to the hue, the brightness level, and opacity. In this situation, the processing circuitry 44 realizing the converting function 445 is an example of a display mode generating unit.

By employing the display controlling function 446, the processing circuitry 44 is configured to cause the display 42 to display images on the basis of the various types of image data generated by the image processing function 443. The images displayed by the display 42 include the color image according to the embodiment. Further, the images displayed by the display 42 include the color superimposed image generated by adding the color image to the base image. Furthermore, the images displayed by the display 42 include a CT image based on the CT image data, a cross-section image based on cross-sectional image data taken on an arbitrary cross-sectional plane, a rendering image in an arbitrary viewpoint direction based on the rendering image data in the arbitrary viewpoint direction, and the like. Further, the images displayed by the display 42 include an image for displaying an operation screen and an image for displaying a notification and an alert for the operator. In this situation, the processing circuitry 44 realizing the display controlling function 446 is an example of a display controlling unit.

The image data of the color superimposed image generated by adding the color image according to the embodiment to the base image and of a display screen including the color superimposed image or the like may be generated by either one of the image processing function 443 and the display controlling function 446.

Further, the functions 441 to 446 do not necessarily have to be realized by a single piece of processing circuitry. It is also acceptable to structure the processing circuitry 44 by combining together a plurality of independent processors, so that the functions 441 to 446 are realized as a result of the processors executing the programs. In this situation, the functions 441 to 446 may be realized as being distributed among or integrated into one or more pieces of processing circuitry, as appropriate.

Although the example was explained in which the console 40 being a single console is configured to execute the plurality of functions, it is also acceptable to have the plurality of functions executed by separate consoles. For example, it is also acceptable to have the functions of the processing circuitry 44 in a distributed manner, namely the image generating function 442, the image processing function 443, the category determining function 444, the converting function 445, and/or the like.

Further, a part or all of the processing circuitry 44 does not necessarily have to be included in the console 40 and may be included in an integration server configured to perform a process collectively on detection data acquired by a plurality of medical image diagnosis apparatuses.

Further, one or more processes selected from among the post-processing process, the generating process, the learning process, the identifying process, and the display process may be performed by either the console 40 or an external workstation. Further, one or more of the processes may be performed simultaneously by both the console 40 and a workstation. As the workstation, for example, it is possible to use, as appropriate, a computer or the like including a processor configured to realize the functions corresponding to the processes and memory elements such as a ROM and a RAM or the like as hardware resources thereof.

Further, to reconstruct the X-ray CT image data, it is possible to adopt either one of the reconstruction schemes between a full scan reconstruction scheme or a half scan reconstruction scheme. For example, by employing the image generating function 442, the processing circuitry 44 is configured, in the full scan reconstruction scheme, to use projection data corresponding to a full circle around the patient P, i.e., 360 degrees. In contrast, in the half scan reconstruction scheme, the processing circuitry 44 is configured to use projection data corresponding to 180 degrees+a fan angle. In the following sections, for the sake of convenience in the explanation, it is assumed that the processing circuitry 44 is configured to use the full scan reconstruction scheme in which the reconstruction is performed by using the projection data corresponding to a full circle around the patient P, i.e., 360 degrees.

The techniques according to the present embodiment are applicable to both an X-ray computed tomography apparatus of a single tube type and an X-ray computed tomography apparatus of a so-called multiple-tube type in which a plurality of pairs each made up of an X-ray tube and a detector are installed on a rotating ring.

Further, the techniques according to the present embodiment are also applicable to the X-ray CT apparatus 1 configured to be able to perform imaging processes by using a dual energy scheme. In that situation, the X-ray high-voltage device 14 is capable of alternately switching between energy spectra of the X-rays emitted from the X-ray tube 11, by performing high-speed switching between two voltage values, for example. In other words, the X-ray CT apparatus 1 may be configured to acquire projection data in each acquisition view, while modulating X-ray tube voltage with timing corresponding to a control signal for modulating the X-ray tube voltage. By imaging the patient while using mutually-different X-ray tube voltage levels, it is possible to enhance dark/light contrast of CT images, on the basis of substance energy transmissibility for each of the X-ray energy spectra.

Further, it is assumed that the X-ray CT apparatus 1 according to the present embodiment is configured to read the electrical signals from the X-ray detector 12 by using a successive reading method.

In an example, the X-ray CT apparatus 1 according to the present embodiment may be configured as a standing CT apparatus. In that situation, in place of moving the tabletop 33, it is possible to provide a patient supporting mechanism configured to support the patient P who is standing and to be movable along the rotation axis of a rotating part of the gantry 10. In another example, the X-ray CT apparatus 1 according to the present embodiment may be configured as a movable CT apparatus in which the gantry 10 and the table 30 are movable.

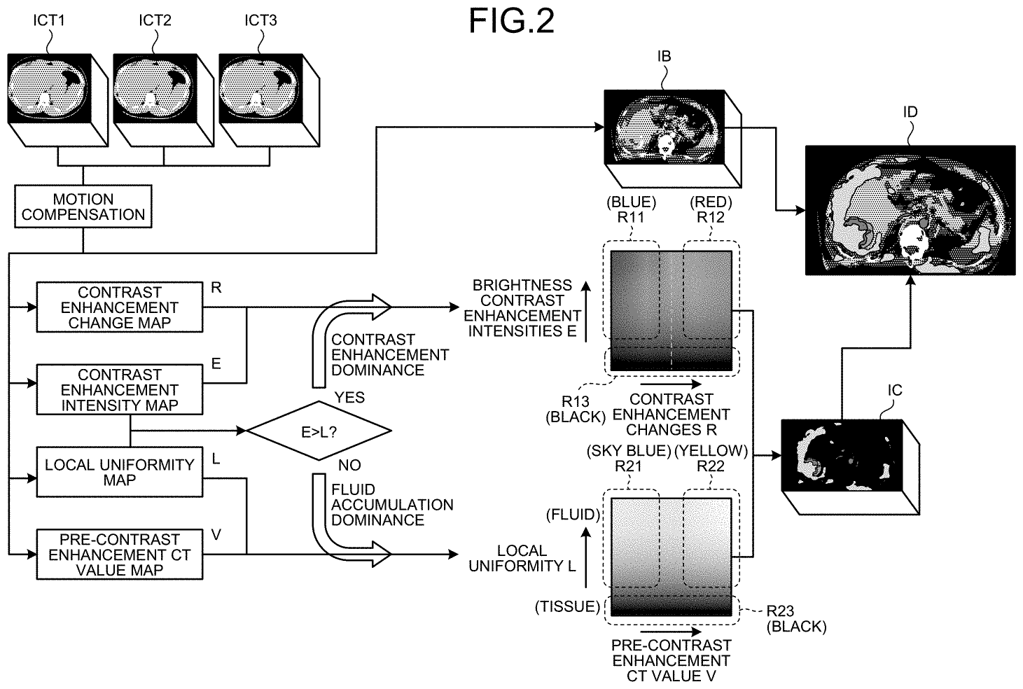

Next, the display mode generating process in the medical image processing process according to the embodiment will be explained further in detail, with reference to drawings. FIG. 2 is a chart for explaining the display mode generating process in the medical image processing process according to the embodiment.

In the following sections, the display mode generating process in the medical image processing process according to the embodiment will be explained by using an example in which contrast agent extravasation and abdominal cavity fluid accumulation are rendered as a color image IC (FIG. 2), on the basis of abdominal contrast-enhanced dynamic CT in the three temporal phases, namely, the pre-contrast enhancement CT (i.e., plain CT), the artery dominant phase contrast-enhanced CT, and the parenchymal phase contrast-enhanced CT.

### 1. Reading Multiple Temporal Phase Images

The system controlling function 441 is configured to read, from the memory 41 for example, image data of a plain CT image ICT1, an abdominal contrast-enhanced dynamic CT image in an artery dominant phase (hereinafter, “artery dominant phase CT image ICT2), and an abdominal contrast-enhanced dynamic CT image in a parenchymal phase (hereinafter, “parenchymal phase CT image ICT3). In this situation, the plain CT image ICT1 is a CT image taken of the range from the chest to the thighs prior to the contrast enhancement. The artery dominant phase CT image ICT2 is a CT image taken after a contrast agent was injected. For example, “after a contrast agent was injected” denotes approximately 30 seconds after the injection of the contrast agent. In another example, “after a contrast agent was injected” may denote a number of seconds after the contrast of the aorta was enhanced, for instance. The parenchymal phase CT image ICT3 is a CT image taken tens of seconds later than the artery dominant phase.

### 2. Designating a Target Range

For example, on the basis of an input operation received from the operator via the input interface 43, the image processing function 443 is configured to select a target region for generating the color image from the read CT images corresponding to the entire range. For example, the target region may be selected from among: the head, the chest, the abdomen, a pelvis region, and the abdomen and pelvis. For example, when the “abdomen” is selected, the image processing function 443 is configured to identify, from the CT images, a slice position 2 centimeters (cm) above an upper diaphragm part and another slice position 10 cm below a lower kidney part and to further determine these slice positions as an upper end position and a lower end position of a processed range in the color image generating process. Further, the image processing function 443 is configured to identify a dorsal surface position of the torso, as well as an abdominal surface position and left and right lateral positions and to further record the identified positions into the memory 41, for example, as a cuboid representing the processed range.

In addition, the image processing function 443 is configured to determine a slice interval and a pixel interval in accordance with the selected target region and to record the intervals into the memory 41, for example.

### 3-1. Motion Compensation

The image processing function 443 is configured to perform motion compensation in relation to the contrast-enhanced images in the different temporal phases. For the motion compensation, it is possible to use, for example, a non-rigid registration algorithm. At first, the image processing function 443 is configured to generate an image obtained by extracting the cuboid part representing the processed range from the plain CT image ICT1. To extract the image, an image interpolation scheme such as cubic spline may be used. The image processing function 443 is configured to generate the image that corresponds to the recorded cuboid representing the processed range and that has the recorded slice and pixel intervals. Subsequently, the image processing function 443 is configured to perform non-rigid registration on the artery dominant phase CT image ICT2, by using the extracted plain CT image ICT1 as a reference image. The image processing function 443 is configured to temporarily record, in the memory 41 for example, a result of the non-rigid registration as a motion-compensated artery dominant phase CT image ICT2. Further, the image processing function 443 is configured to perform non-rigid registration on the parenchymal phase CT image ICT3, by using the extracted plain CT image ICT1 as a reference image. The image processing function 443 is configured to temporarily record, in the memory 41 for example, a result of the non-rigid registration as a motion-compensated parenchymal phase CT image ICT3. The extracted plain CT image ICT1 is temporarily recorded in the memory 41, for example, as a motion-compensated plain CT image ICT1, without applying any modification thereto.

### 3-2. Applying Image Filter

The image processing function 443 is configured to apply an image filter to the plain CT image ICT1, to the artery dominant phase CT image ICT2, and to the parenchymal phase CT image ICT3 that were motion-compensated. In an example, the image processing function 443 is configured to apply either a Gaussian filter or a diffusion filter to each of the images, to generate the plain CT image ICT1, the artery dominant phase CT image ICT2, and the parenchymal phase CT image ICT3 that were motion-compensated. The image processing function 443 is configured to temporarily record the generated images into the memory 41 for example, as the plain CT image ICT1, the artery dominant phase CT image ICT2, and the parenchymal phase CT image ICT3 that were motion-compensated and filtered.

4. Generating Images related to Spatial or Inter-Temporal Phase Changes in Pixel Values

The image processing function 443 is configured to calculate, with respect to each of the pixels for example, a plurality of variable values related to spatial changes or inter-temporal phase changes in the pixel values, on the basis of the CT images ICT1, ICT2, and ICT3 corresponding to the plurality of temporal phases, as described below. In this situation, the image processing function 443 may construct and hold therein a plurality of images from the calculated variable values or may perform the processes up to the conversion into a color coding image in units of pixels without holding the images therein.

### 4-1. Generating Contrast Enhancement Intensity Map

The image processing function 443 is configured to generate a contrast enhancement intensity map E. The contrast enhancement intensity map E is an image in which each of the pixels has a value expressing a contrast enhancement intensity. The contrast enhancement intensity map E is calculated by using Expression (1) presented below:

E=max(S1−S0, S2−S0) (1)

In the above expression, S0, S1, and S2 each denote the CT value of each of the pixels in the plain CT image ICT1, the artery dominant phase CT image ICT2, and the parenchymal phase CT image ICT3 that were motion-compensated and filtered, respectively. When any of the CT values among S0, S1, and S2 is outside a CT value range of parenchymal organs, the pixel is determined to satisfy E=−2048. In this situation, −2048 is a padding value indicating a value to be ignored in subsequent processing.

### 4-2. Generating Contrast Enhancement Change Map

The image processing function 443 is configured to generate a contrast enhancement change map R. The contrast enhancement change map R is an image in which each of the pixels has a value expressing a contrast enhancement change amount in a late phase. The contrast enhancement change map R is calculated by using Expression (2) presented below:

R=100×(S2−S1)/max(E, 1) (2)

In the above expression, S0, S1, and S2 each denote the CT value of each of the pixels in the plain CT image ICT1, the artery dominant phase CT image ICT2, and the parenchymal phase CT image ICT3 that were motion-compensated and filtered, respectively. When any of the CT values among S0, S1, and S2 is outside the CT value range of parenchymal organs, the pixel is determined to satisfy R=0.

### 4-3. Generating Local Uniformity Map

The image processing function 443 is configured to generate a local uniformity map L. The local uniformity map L is an image in which each of the pixels has a value expressing local uniformity L. The image processing function 443 is configured to generate an image in which each of the pixels has the difference between a maximum value image and a minimum value image among the plain CT image ICT1, the artery dominant phase CT image ICT2, and the parenchymal phase CT image ICT3 that were motion-compensated and filtered and to further generate the local uniformity map L from the image indicating local standard deviations. The local standard deviation image is an image in which, with respect to an arbitrary pixel, the pixel value expresses a standard deviation from the pixels in the surroundings thereof (e.g., within a 5-mm radius). The local uniformity map L is calculated by applying a mathematical function (e.g., a reciprocal or exponential mathematical function) of constant multiple of the variance image (square of local standard deviation image). A region having a high local uniformity value is a region having a possibility of fluid accumulation. When the local uniformity is used for evaluating a likelihood of fluid accumulation, the local uniformity dominance denotes fluid accumulation dominance. In the following sections, an example will be explained in which the local uniformity dominance is used as the fluid accumulation dominance.

### 4-4. Generating Pre-Contrast Enhancement CT Value Map

The image processing function 443 is configured to generate a pre-contrast enhancement CT value map V. The pre-contrast enhancement CT value map V is an image in which each of the pixel has a characteristic relevant to the pixel value in the pre-contrast enhancement CT image ICT1. The image processing function 443 is configured to use the plain CT image ICT1 that was motion-compensated and filtered as the pre-contrast enhancement CT value map V, without applying any modification thereto.

### 4-5. Generating Mask Image

The image processing function 443 is configured to generate a mask image C. The mask image C is an image in which the pixel values in a region to be rendered as a color image are each determined to be 1, whereas the pixel values in a region not to be rendered in the image are each determined to be 0. Most simply, the image processing function 443 is configured to generate the mask image C in which the pixel value is determined to be 0 when S0, S1, and S2 are outside the CT value range of parenchymal organs, whereas the pixel value is determined to be 1 when S0, S1, and S2 are within the CT value range of parenchymal organs.

5. Conversion into Color Coding Image

The converting function 445 is configured to generate the color image IC by using a plurality of color value mathematical functions, on the basis of the four images that are related to the spatial changes or the inter-temporal phase changes in the pixel values and were generated by the image processing function 443 and the categories to which the pixels in the images are determined to belong by the category determining function 444.

### 5-1. Normalizing Pixel Values

The category determining function 444 is configured to normalize the pixel values in the five images E, R, L, V, and C by using respective typical lower and upper limit values, as indicated in Expressions (3) to (7), respectively:

$${E}_{n} = min ( 1.0 , max ( 0 , \frac{E - {E}_{min}}{{E}_{max} - {E}_{min}} ) ) \qquad ( 3 )$$

$${R}_{n} = min ( 1 \text{.0} , max ( 0 , \frac{R - {R}_{min}}{{R}_{max} - {R}_{min}} ) ) \qquad ( 4 )$$

$${L}_{n} = min ( 1.0 , max ( 0 , \frac{L - {L}_{min}}{{L}_{max} - {L}_{min}} ) ) \qquad ( 5 )$$

$${V}_{n} = min ( 1.0 , max ( 0 , \frac{V - {V}_{min}}{{V}_{max} - {V}_{min}} ) ) \qquad ( 6 )$$

$${C}_{n} = min ( 1.0 , max ( 0 , \frac{C - {C}_{min}}{{C}_{max} - {C}_{min}} ) ) \qquad ( 7 )$$

In the above expressions, Emin, Emax, Rmin, Rmax, Lmin, Lmax, Vmin, Vmax, Cmin, and Cmax denote a lower limit value of the contrast enhancement intensity map E, an upper limit of the contrast enhancement intensity map E, a lower limit value of the contrast enhancement change map R, an upper limit value of the contrast enhancement change map R, a lower limit value of the local uniformity map L, an upper limit value of the local uniformity map L, a lower limit value of the pre-contrast enhancement CT value map V, an upper limit value of the pre-contrast enhancement CT value map V, a lower limit value of the mask image C, and an upper limit value of the mask image C, respectively.

### 5-2. Determining Categories

The category determining function 444 is configured to determine to which of the two categories each of the pixels belongs, on the basis of at least one of the pixel values En, Rn, Ln, Vn, and Cn from the five images. In this situation, the contrast enhancement intensity map E and the contrast enhancement change map R are each an image based on temporal changes in the data value of each of the pixels in the contrast-enhanced images corresponding to the plurality of temporal phases.

In the simplest example, the category determining function 444 is configured to classify each of the pixels in one of the two categories “local uniformity dominance” and “contrast enhancement dominance”. A pixel exhibiting local uniformity dominance is a pixel exhibiting that fluid accumulation is dominant. More specifically, when En>Ln is satisfied, the category determining function 444 is configured to determine that the pixel belongs to the contrast enhancement dominance category. On the contrary, when En Ln is satisfied, the category determining function 444 determines that the pixel belongs to the local uniformity dominance category. Alternatively, when En =Ln is satisfied, the pixel may be determined to belong to the contrast enhancement dominance category.

### 5-3. Determining Hue and Brightness of Each of Pixel

The converting function 445 is configured to determine a hue and brightness of each of the pixels, by using color conversion mathematical functions each of which is associated with a different one of the categories determined by the category determining function 444.

For example, with respect to each of the pixels exhibiting contrast enhancement dominance, the converting function 445 is configured to calculate a hue h, brightness b, and opacity a by using Expressions (8) to (10), respectively.

h=hue_{E}(Rₙ) (8)

b=max(Eₙ, 0)×Cₙ (9)

α=b (10)

For example, with respect to each of the pixels exhibiting local uniformity dominance, the converting function 445 is configured to calculate a hue h, brightness b, and opacity α by using Expressions (11) to (13), respectively.

h=hue_{L}(Vₙ) (11)

b=max(Lₙ, 0)×Cₙ (12)

α=b (13)

In the above expressions, y=hueE(x) and y=hueL(x) are each a hue mathematical function for calculating a hue y from a pixel value x. A color code to be assigned to each of the pixels is calculated as the product of the hue h and the brightness b. The conversion of a pixel value into the color code may be implemented as a Look-Up Table (LUT) used for obtaining the color code by referring to the table. The two color conversion mathematical functions associated with the two categories, respectively, may be implemented as a mathematical function for generating the color image IC from partial spaces of four variables. In the situations where the contrast enhancement dominance is exhibited, the color code is determined from three variables R, E, and C, as indicated in Expressions (8) to (10). Similarly, in the situations where the local uniformity dominance is exhibited, the color code is determined from three variables V, L, and C, as indicated in Expressions (11) to (13). The opacity a is used at the time of displaying an image by volume rendering.

The converting function 445 is configured to generate the color image IC in which each of the pixels has the values expressing a hue, brightness, and opacity, in accordance with the color codes determined in the manner described above. The color conversion mathematical functions are each defined so that the colors are continuous in a connection part (boundary) between the contrast enhancement dominance and the local uniformity dominance, while the hues are different between the categories. FIG. 2 illustrates an example using the two hue mathematical functions, hue E() and hue L().

According to the hue mathematical function hue E() for the contrast enhancement dominance, as observed in the region R11, when the contrast enhancement change R is a negative value, i.e., when the pixel value in the parenchymal phase is smaller than that in the artery dominant phase, blue is assigned. In contrast, as observed in the region R12, when the contrast enhancement change R is a positive value, i.e., when the pixel value in the parenchymal phase is larger than that in the artery dominant phase, red is assigned. Further, as observed in a region between the region R11 and the region R12, when the contrast enhancement change R is near 0, i.e., when the pixel value in the parenchymal phase is approximately equal to that in the artery dominant phase, purple, which is an intermediate color, is assigned.

In other words, in the example of FIG. 2, pixels each having a larger pixel value in the artery dominant phase are displayed in blue when the contrast enhancement intensity decreases in the parenchymal phase and are displayed in red when the contrast enhancement intensity is further intensified toward the parenchymal phase. Accordingly, it is understood that regions displayed in blue, purple, and red each represent either a blood vessel or a contrast agent extravasation image. Further, for the contrast agent extravasation image, it is possible to speculate from the hues whether the contrast agent extravasation is due to hemorrhage from an artery or due to hemorrhage from the portal vein or a vein.

Further, according to the hue mathematical function hue L() for the local uniformity dominance, as observed in the region R21, when the pixel value in the plain CT is small, sky blue is assigned. In contrast, as observed in the region R22, when the pixel value in the plain CT is large, yellow is assigned. Further, as observed in a region between the region R21 and the region R22, when the pixel value in the plain CT is near the middle, green, which is an intermediate color, is assigned.

It is known that, when fluid accumulation is present, CT values of a hematoma are larger than CT values of abdominal fluid (ascites). Accordingly, in the example of FIG. 2, it is possible to speculate that there is a high possibility that the fluid accumulation is consisted of ascites when the displayed color is closer to sky blue and that there is a high possibility that the fluid accumulation is consisted of a hematoma when the displayed color is closer to yellow.

The two hue mathematical functions are defined so that there are no duplication of hue in the two functions. Accordingly, on the basis of the hues in the generated image, the user is able to easily determine whether each region represents a contrast agent extravasation image or a fluid accumulation on the basis of the displayed colors. Although hue mathematical functions are usually configured to output colors having a high chroma, it is also acceptable to cause colors having a low chroma to be output in a partial section thereof. In that situation, by treating the colors having a low chroma as “no hue defined”, it is possible to ensure that those colors are not duplicate of other colors having a high chroma.

Further, even when there is no discontinuous part in the pixel values in an original image, so that the pixel values in the original image are spatially smooth, the color image IC generated by the method described above exhibits, strictly speaking, discontinuous color codes in the part corresponding to En=Ln. However, as explained above, the variable values are defined so that the value in the local uniformity L is small, when the value in the contrast enhancement intensity map E is large. Accordingly, in the display mode generating process according to the present embodiment, there is no possibility that both E and L have large values. More specifically, in a region where En≈Ln is satisfied, brightness values are small in both the contrast enhancement dominance region and the local uniformity dominance region. Accordingly, as observed in the regions R13 and R23 in FIG. 2, in the generated color image IC, both of the regions are colored close to black, so that it is possible to consider that the colors in the color image IC are continuous with the black color.

As explained above, the variable calculation expressions, the category determining method, and the color conversion mathematical functions according to the present embodiment are each defined so that the colors in the color image IC have substantially no spatial discontinuity, as long as the pixel values in the original image are spatially continuous. In other words, the color conversion mathematical function corresponding to the contrast enhancement dominance category and the color conversion mathematical function corresponding to the fluid accumulation dominance category are defined so as to convert the value of at least one variable value into the same color code. With this configuration, with respect to regions where CT pixel values in the original image have small spatial changes, it is possible to prevent even the color image IC from exhibiting large spatial changes. In other words, when viewing a part where the colors are discontinuous in the color image IC, image interpreters are able to determine that the pixel values in the original image are discontinuous. It is possible to determine that the color discontinuity in the color image IC anatomically expresses a boundary between one of types, structures, and properties of the tissue. Consequently, an advantageous effect is achieved where image interpretation is made easier by defining the colors in the color image IC have substantially no spatial discontinuity. Generating Base Image

The image processing function 443 is configured to generate, as a base image IB, an inter-temporal phase maximum value image among the plain CT image ICT1, the artery dominant phase CT image ICT2, and the parenchymal phase CT image ICT3 that were motion-compensated, so as to temporarily record the generated base image IB in the memory 41, for example.

In this situation, instead of the inter-temporal phase maximum value image, the image processing function 443 may generate, as the base image IB, one selected from among the plain CT image ICT1, the artery dominant phase CT image ICT2, and the parenchymal phase CT image ICT3 that were motion-compensated.

### Outputting the Image

The image processing function 443 is configured to generate a color superimposed image ID by adding the color image IC to the base image IB. The display controlling function 446 is configured to output the generated color superimposed image ID to the display 42 so as to be displayed on the display 42. Alternatively, the image processing function 443 may output the generated color superimposed image ID to the memory 41 for example, as an image record. For example, the image processing function 443 may be configured to collectively output images on a plurality of cross-sections as a plurality of images. In another example, the image processing function 443 may generate a multiple cross-section display image in which the cross-section images are arranged in the single image in a matrix formation.

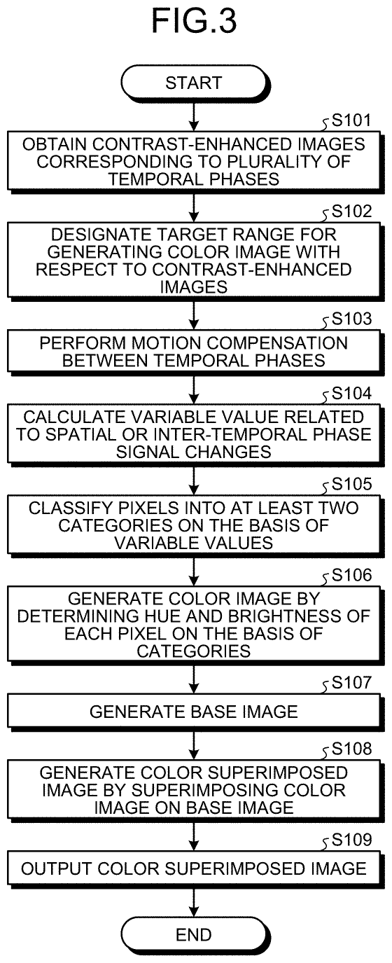

Next, a flow in the display mode generating process in the medical image processing process according to the embodiment will be explained, with reference to the drawings. FIG. 3 is a flowchart illustrating an example of the medical image processing process according to the embodiment.

To begin with, the system controlling function 441 obtains the contrast-enhanced images corresponding to the plurality of temporal phases from the memory 41, for example (step S101). As the contrast-enhanced images corresponding to the plurality of temporal phases, for example, the plain CT image ICT1, the artery dominant phase CT image ICT2, and the parenchymal phase CT image ICT3 may be obtained, as explained above.

On the basis of an input operation received from the operator via the input interface 43, for example, the image processing function 443 designates a target range for generating the color image with respect to the obtained contrast-enhanced images corresponding to the plurality of temporal phases (step S102). Further, the image processing function 443 performs the motion compensation between the temporal phases and the filtering process (step S103).

From the contrast-enhanced images corresponding to the plurality of temporal phases, the image processing function 443 calculates, with respect to each of the pixels, a variable value related to a spatial or inter-temporal phase signal change (step S104). In this situation, the variable value related to the spatial or inter-temporal phase signal change denotes a variable value related to either a spatial change or an inter-temporal phase change in the pixel value. More specifically, the image processing function 443 generates the contrast enhancement intensity map E, the contrast enhancement change map R, the local uniformity map L, the pre-contrast enhancement CT value map V, and the mask image C.

On the basis of the calculated variable values, i.e., an image related to either the spatial changes or the inter-temporal phase changes in the pixel values, the category determining function 444 classifies each of the pixels into one of at least two categories (step S105).

The converting function 445 generates a color image by determining a hue and brightness of each of the pixels on the basis of the determined categories (step S106). More specifically, the converting function 445 generates the color image, by determining a color code of each of the pixels while using one of the color conversion mathematical functions corresponding to the category to which the pixel belongs.

For example, the image processing function 443 generates, as the base image IB, a maximum value image among the temporal phases, as compared among the plain CT image ICT1, the artery dominant phase CT image ICT2, and the parenchymal phase CT image ICT3 that were motion-compensated (step S107).

The image processing function 443 generates a color superimposed image obtained by superimposing the color image on the base image (step S108). Further, the display controlling function 446 outputs the generated color superimposed image to the display 42, for example, so as to cause the display 42 to display a display screen including the color superimposed image (step S109).

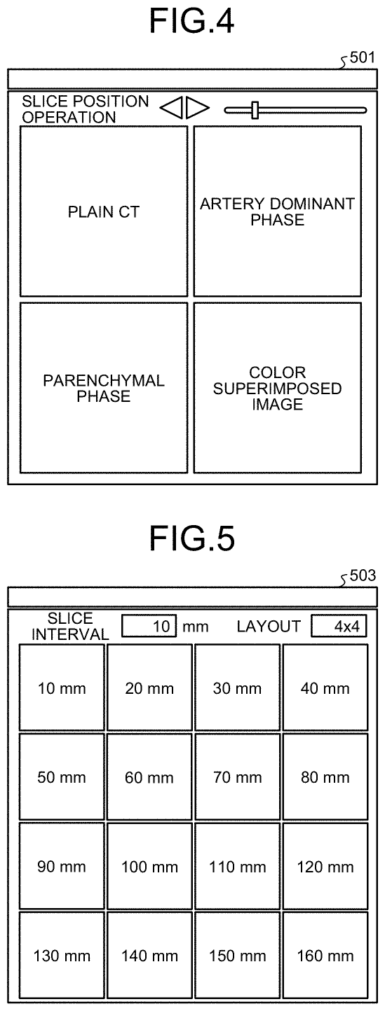

FIG. 4 is a drawing illustrating an example of the display modes generated in the medical image processing process according to the embodiment. FIG. 4 illustrates a display screen 501 including the generated color superimposed image. As illustrated in FIG. 4, the display controlling function 446 is configured to cause the display 42 to display the display screen 501 including the contrast-enhanced images corresponding to the plurality of temporal phases and the color superimposed image. Further, the display controlling function 446 may display a slider or the like used for instructing a slice position operation and is also capable of displaying images related to a slice position corresponding to the user operation.

As explained above, the display modes generated by the medical image processing apparatus according to the present embodiment includes the display of a list of the information related to a plurality of characteristics such as the CT values, the contrast enhancement intensities, the contrast enhancement signal changes, and the fluid accumulation. Consequently, because a distribution of contrast agent extravasation and fluid accumulation due to hemorrhage is displayed in color in the single image, the image interpreter is able to easily recognize a damaged blood vessel having contrast agent extravasation and the presence/absence of leakage to the outside of the organ. Further, the image interpreter is able to easily recognize whether or not the fluid accumulation is a hematoma and to easily determine the presence/absence of hemorrhage and the amount thereof.

FIG. 5 is a drawing illustrating another example of the display modes generated in the medical image processing process according to the embodiment. FIG. 5 illustrates a display screen 503 including the generated color superimposed image. As illustrated in FIG. 5, the display controlling function 446 is configured to cause the display 42 to display a display screen 503 including color superimposed images on multiple cross-sections. Further, the display controlling function 446 may display an input window or the like used for instructing a slice interval or the number of images to be displayed at once and is also capable of displaying the images at the slice interval and in the quantity corresponding to the user operation. Further, the display controlling function 446 is also capable, in response to a user operation, of switching the display among the display screen including the multiple cross-section color superimposed images, a display screen including multiple cross-section artery dominant phase CT images, and a display screen including multiple cross-section parenchymal phase CT images.

As explained above, the display modes generated by the medical image processing apparatus according to the present embodiment include the multiple cross-section display of the color superimposed images. As a result, the image interpreter is able to more quickly recognize the damaged blood vessel having the contrast agent extravasation and the presence/absence of leakage to the outside of the organ.

Generally speaking, abdominal contrast-enhanced dynamic CT is performed in the situation where, in an initial diagnosing process for a patient who was transported in emergency due to an abdominal injury or other injuries, there is no heavy hemorrhage, and the condition is relatively stable without any central nervous system disorder, while damage to an organ in the abdominal cavity is suspected or in the situation where plain CT discovered only small hemorrhage in the abdominal cavity and no damage in tubular organs. The abdominal contrast-enhanced dynamic CT may be performed for the purpose of determining whether there is heavy hemorrhage, whether parenchymal organs are heavily damaged, and whether tubular organs are damaged. When it is determined that there is heavy hemorrhage, one or more parenchymal organs are heavily damaged, or one or more tubular organs are damaged, laparotomy surgery will be selected. Further, when the blood vessel having hemorrhage is an artery, treatment involving Trans Arterial Embolization (TAE) is considered, so as to explore a treatment approach such as examining an artery to be embolized on the basis of a contrast-enhanced CT image. In contrast, when the hemorrhage is absent or relatively small, a non-surgical treatment method will be selected.

To reach the above determination by using contrast-enhanced CT, it is important to analyze leakage of the contrast agent to the outside of blood vessels (a contrast agent extravasation image), to evaluate the presence/absence of a hematoma in the abdominal cavity, and to evaluate the volume of the hematoma in the abdominal cavity. In the contrast agent extravasation image, important information includes which blood vessel is the damaged blood vessel, for example, which blood vessel among arteries, the portal vein, and veins is the damaged blood vessel. Further, other important information includes whether the blood leaking from a blood vessel has reached to the outside of the organ such as the abdominal cavity or the like to exhibit active hemorrhage and whether the speed of the hemorrhage is high. Also, other important information in finding out the amount of hemorrhage includes whether or not fluid is accumulating in the abdominal cavity, whether the accumulating fluid is a hematoma or ascites, and how much volume the hematoma has. In addition, to determine necessity for restoring the organ and a method therefor, an evaluation is made on the manner, the position, the extent, and the like of the damage to the organ.

In an example, to reach the above determination, among contrast-enhanced dynamic CT schemes, CT images such as a plain CT image which is non-contrast enhanced CT prior to contrast enhancement, an artery dominant phase CT image, and a parenchymal phase CT image may be used. Conventionally, image interpreters have been using an image viewer to display these three types of images corresponding to a plurality of temporal phases, so as to make a visual comparison to analyze a contrast agent extravasation image and abdominal cavity fluid accumulation.

However, to accurately understand the signal value changes caused by contrast agent extravasation, it is necessary to compare in detail and observe the images corresponding to the plurality of temporal phases. It has therefore been difficult to promptly and accurately reach the determination.

In another example, because contrast agent extravasation causes changes in pixel values in contrast-enhanced dynamic CT, it is possible to display the changes in the pixel values by using a mode such as a color image. For example, in a difference image between an artery dominant phase and plain CT, arteries are primarily rendered with high signals and, if there is any hemorrhage from an artery, contrast agent extravasation caused thereby is also expected to be rendered. The image in this example is an initial contrast-enhanced image reflecting the extent of contrast enhancement at an initial stage. In an actual calculation, a signal ratio between the artery dominant phase and the plain CT may be used instead of the difference. Further, in a difference image between a parenchymal phase and an artery dominant phase, veins are rendered with high signals, whereas arteries are rendered with low signals (negative values), while the manner in which hemorrhage from an artery is spreading afar and hemorrhage from a vein are expected to be rendered. The image in this example is a signal change image reflecting signal changes in a later phase. These difference images and parametric images, which are calculation images similar to the difference images, are often displayed while being colored, i.e., while being color coded. It is also possible to use a peak contrast enhancement intensity image obtained by subtracting signal values of plain CT from the higher of the signal values between an artery dominant phase and a parenchymal phase. Further, because movements of the patient during the imaging make it impossible to obtain an accurate parametric image, motion compensation is requisite. The color-coded parametric image may be displayed while being superimposed on an original CT image.

For example, as a result of superimposing an initial contrast-enhanced image as a color image on a plain CT image, it is possible to observe contrast enhancement between a pre-contrast enhancement phase and an artery dominant phase. Further, as a result of superimposing a signal change image as a color image on a plain CT image, it is possible to observe contrast enhancement signal changes from an artery dominant phase to a parenchymal phase. As explained herein, as a result of displaying the parametric image in the superimposed manner, because it is possible to obtain the necessary information by simply comparing two images visually, the image interpretation is easier than in the situation where the images in the three temporal phases of dynamic CT need to be visually compared. However, because it is still necessary to visually compare in detail and observe the two images, it has been difficult to reach the determination promptly and accurately.

As yet another example, a technique has been proposed by which a blood vessel region, a contrast agent having extravasation, and a fluid accumulation region can be extracted. For example, it is possible to extract, most simply, the blood vessel region and the fluid accumulation region by using a threshold value process or a region growing method. The regions extracted in this manner may be superimposed in color on an original CT image. For example, a technique is known by which a blood vessel region, a contrast agent extravasation region, and a fluid accumulation region are displayed in a translucent manner, on a CT image in an artery dominant phase. An alpha blending method may be used, for example, for the translucent superimposition display. In volume rendering, it is possible to handle a plurality of types of extracted regions as multi-object elements, so as to be displayed in mutually-different color tones.

However, when the region extracting technique is used for the blood vessel region, for the contrast agent extravasation region, or for the fluid accumulation region, there is a possibility that the region may be erroneously extracted. If erroneous extractions occur more frequently than a predetermined level, it will be difficult to perform the image interpretation even though there is no need to visually compare the plurality of images with one another. Generally speaking, blood vessels are slenderer in distal parts, and the signal changes caused by the contrast enhancement also become smaller due to the partial volume effect. For these reasons, it is considered that the periphery of blood vessels has a high possibility of being missed from the extraction. Further, false contrast enhancement caused by movements of the patient can also be a cause of erroneous extractions. As for the contrast agent extravasation region, because there is a large variation since extravasation regions are not so simple as blood vessels and because the changes in the pixel values may be small in some situations, it is difficult to extract a reliable region. The region having fluid accumulation in the abdominal cavity is a range where the signal values correspond to blood or fluid and can therefore be extracted as a region having no signal changes caused by the contrast enhancement. However, because regions having no signal changes caused by the contrast enhancement do not necessarily correspond only to fluid accumulation in the abdominal cavity, it has been difficult to accurately determine such a region.

Further, when the extracted region is superimposed on an original image, there has been a problem where it is not possible to find out the signal values or the extent of the signal changes. For example, when a contrast agent extravasation region is superimposed in color on an original image, although it is possible to understand the range of the extravasation, it is not possible to understand signal values of the extravasation or the magnitude of the signal changes. The magnitude of the signal changes caused by the extravasation serves as an important clue for determining whether the damaged blood vessel is an artery or a vein. Accordingly, when the extract region is displayed as being superimposed in color on the original image, it has been difficult to reach accurate determination, because of the problem of the erroneous extraction and because of the problem where the information about the signal values and the signal changes is missing.

To cope with the circumstances described above, in the X-ray CT apparatus 1 having installed therein the medical image processing apparatus according to the present embodiment, the processing circuitry 44 is configured to be able to realize the system controlling function 441, the category determining function 444, and the converting function 445. The system controlling function 441 is configured to obtain the contrast-enhanced images related to the patient P and corresponding to the plurality of temporal phases. On the basis of the data values of the pixels in the contrast-enhanced images corresponding to the plurality of temporal phases, the category determining function 444 is configured to determine which one of contrast enhancement dominance and fluid accumulation dominance corresponds to each of the pixels or each group of pixels. The converting function 445 is configured to generate the display modes based on a result of the determination by the category determining function 444.

With this configuration, it is possible to generate the display modes for displaying the color superimposed image, which corresponds to visually comparing the contrast-enhanced images in the plurality of temporal phases with one another. Consequently, it is possible to generate the display modes that contribute to performing a medical image diagnosing process in the contrast-enhanced dynamic examination more accurately and more promptly than in the situation where the plurality of images are visually compared with one another.

### Second Embodiment

In the present embodiment, differences from the first embodiment will primarily be explained. The display controlling function 446 may cause the display 42 to display a display screen including, not only the color superimposed image, but a color image.

For example, similarly to the display screen 501, the display controlling function 446 is also capable of causing the display 42 to display a display screen including the contrast-enhanced images corresponding to the plurality of temporal phases and a color image.

In another example, similarly to the display screen 503, the display controlling function 446 is also capable of causing the display 42 to display a display screen including multiple cross-section color images. In addition, the display controlling function 446 is also capable, in response to a user operation, of switching the display among the display screen including the multiple cross-section color images, a display screen including multiple cross-section artery dominant phase CT images, and a display screen including multiple cross-section parenchymal phase CT images. Furthermore, in response to a user operation, the display controlling function 446 may switch the display to a display screen including multiple cross-section color superimposed images.

In the display mode generating process according to the present embodiment, the base image generating process at step S107 and the color superimposed image generating process at step S108 in FIG. 3 may be performed, but do not necessarily have to be performed.

### Third Embodiment

In the present embodiment, differences from the first embodiment will primarily be explained. In a display mode generating process according to the present embodiment, when the color image or the color superimposed image is displayed, the display related to each of the plurality of categories may individually be turned on/off.

For example, when the local uniformity dominance category is to be turned off, the converting function 445 is configured to cause the brightness value to be 0 with respect to any of the pixels determined to belong to the local uniformity dominance. Alternatively, the converting function 445 may be configured to cause the opacity value to be 1, with respect to any of the pixels determined to belong to the local uniformity dominance. In these situations, the color superimposed image is generated as an addition image of a gray scale image of the base image and a color image of the contrast enhancement dominance.

Further, whether each of the categories is turned on/off may be determined, for example, in accordance with a result of the user's operation via a GUI to turn on/off the color image for each category. In this situation, the converting function 445 is configured to change the brightness or opacity value of each of the pixels in accordance with the operation to turn on/off the color image for each of the categories. Further, the display controlling function 446 is configured to update the display screen in accordance with the operation to turn on/off the color image for each of the categories.

Alternatively, the turning on/off of each of the categories may be determined on the basis of on/off information set in advance, for example. The on/off information may be set in accordance with imaged body sites or the like. In that situation, the converting function 445 is configured to generate color images with respect to the categories set to be ON. Further, the image processing function 443 is configured to generate a color superimposed image obtained by superimposing a color image on a base image, with respect to the categories set to be ON.

Further, when the turning on/off each of the categories is determined on the basis of the on/off information set in advance, the image processing function 443 is also capable of generating and outputting a plurality of color image sets corresponding to multiple combinations related to the on/off states of the categories configured in the on/off information.

### Fourth Embodiment

In the present embodiment, differences from the first embodiment will primarily be explained. In the first embodiment, the example was explained in which the category to which each of the pixels belongs is determined on the basis of the calculated variable values, so as to generate the display modes in which the color codes of the pixels are determined by using the color conversion mathematical functions associated with the determined categories; however, possible embodiments are not limited to this example. In other words, although the example was explained in which each of the pixels is determined to belong to one of the categories as a discrete value (a nominal variable), possible embodiments are not limited to this example.

The converting function 445 according to the present embodiment corresponds to the category determining function 444 and the converting function 445 according to the above embodiments.

With respect to each of the pixels, the converting function 445 is configured to convert the variable value of each of the pixels into a color code, by using a color conversion mathematical function that has applied thereto a class determination method for expressing the intensity of belonging to each category with continuous values.

In an example, as indicated in Expressions (14) to (16), the converting function 445 is configured to convert the variable value of each of the pixels into a color code, by using a color conversion mathematical function corresponding to the contrast enhancement dominance.

h_{E}=hue_{E}(Rₙ) (14)

b_{E}=max(Eₙ, 0)×Cₙ (15)

α_{E}=b_{E} (16)

Further, as indicated in Expressions (17) to (19), the converting function 445 is configured to convert the variable value of each of the pixels into a color code, by using a color conversion mathematical function corresponding to the local uniformity dominance.

h_{L}=hue_{L}(Vₙ) (17)

b_{L}=max(Lₙ, 0)×Cₙ (18)

α_{L}=b_{L} (19)

After that, by using b_{E} in Expression (15) as a membership mathematical function corresponding to the contrast enhancement dominance and using B_{L} in Expression (18) as a membership mathematical function corresponding to the local uniformity dominance, the converting function 445 is configured, as indicated in Expression (20), to generate a color image by calculating a weighted sum of color values while using the membership mathematical functions as weights.

color=b_{E}h_{E}+b_{L}h_{L} (20)

As explained above, in the display mode generating process in the present embodiment, similarly to the first embodiment, the individual color conversion mathematical functions are applied to the plurality of categories; however, in the display mode generating process in the present embodiment, each of the pixels is not explicitly classified into any of the categories. Instead, in the display mode generating process in the present embodiment, the final colors are determined by calculating the weighted sum, while using the colors obtained from the color conversion mathematical functions in correspondence with the categories, as the membership mathematical functions. In other words, it is possible to express that the process in the present embodiment of generating the color image by calculating the weighted sum of the color values while using the membership mathematical functions as the weights corresponds to the process of determining the category to which each of the pixels belongs in the first embodiment.

Similarly to the first embodiment, the color conversion mathematical functions corresponding to the categories are configured so that the hues are not the same between the categories. Further, similarly to the first embodiment, as long as the pixel values in the original image are spatially continuous, the colors in the obtained color image are also spatially continuous.

### Fifth Embodiment

In the present embodiment, differences from the first embodiment will primarily be explained.

In a display mode generating process in the present embodiment, the image processing function 443 is configured to perform volume rendering, by using a color image generated in the same manner as in the first embodiment and an opacity image. In this situation, the opacity image is an image having opacity values each serving as a variable value calculated with respect to each of the pixels.

By performing the volume rendering on the color image in this manner, it is possible, similarly to the example in FIG. 2, to display a three-dimensional image in which contrast enhancement intensities and late-phase contrast enhancement change amounts are displayed in colors from blue to red, whereas the pixel values in the plain CT in a locally uniform region are displayed in colors from sky blue to yellow. As a result, an advantageous effect is achieved where the image interpreter is able to easily speculate a damaged blood vessel, by three-dimensionally understanding a positional relationship between blood vessels and a contrast agent extravasation image. Further, another advantageous effect is also achieved where the image interpreter is able to easily check to see whether fluid accumulation is a hematoma and to thus determine the presence/absence a hematoma and the volume thereof.

### Sixth Embodiment

In the present embodiment, differences from the first embodiment will primarily be explained. The color image in the first embodiment may be generated by using a multi-object scheme.

More specifically, it is also possible to generate the color image by using the following processing procedure being equivalent to that in the first embodiment.

The category determining function 444 is configured to extract a contrast enhancement region and a local uniformity region, by using the motion-compensated contrast-enhanced CT image and the generated local uniformity map L. In the simplest method, the category determining function 444 is configured to extract a region in which the contrast enhancement intensities are equal to or higher than a predetermined value as the contrast enhancement region and to extract a region in which the local uniformity levels are equal to or higher than a predetermined value as the local uniformity region. It is assumed that the threshold values for the contrast enhancement intensities and the local uniformity levels are determined in advance and recorded in the memory 41, for example.

For example, as color conversion mathematical functions corresponding to the contrast enhancement region, the converting function 445 is configured to use the color conversion mathematical functions presented in Expressions (21) and (22).

h_{E}=hue_{E}(Rₙ) (21)

α_{E}=b (22)

Further, for example, as color conversion mathematical functions corresponding to the local uniformity region, the converting function 445 is configured to use the color conversion mathematical functions presented in Expressions (23) and (24).

h_{L}=hue_{L}(Vₙ) (23)

α_{L}=b_{L} (24)

In this situation, with respect to each of the pixels, it is determined which category the pixel belongs, similarly to the first embodiment. Further, the color conversion mathematical functions respectively applied to the plurality of categories are, similarly to the first embodiment, individual color conversion mathematical functions corresponding to the categories. Furthermore, similarly to the first embodiment, the color conversion mathematical functions corresponding to the categories are configured so that the hues are not the same between the categories. In addition, similarly to the first embodiment, as long as the pixel values in the original image are spatially continuous, the colors in the obtained color image are also spatially continuous.

Generally speaking, many of volume rendering software applications (library) have a multi-object function so as to be able to determine a color and an opacity level, by applying one of mutually-different LUTs to each of the segmented regions. Accordingly, it is possible to implement the display mode generating process according to the present embodiment, by using an ordinary volume rendering library.

### Seventh Embodiment

In the present embodiment, differences from the fifth embodiment will primarily be explained. A display mode generating process according to the fifth embodiment is also applicable to projection images.

For example, the image processing function 443 is configured to obtain a plurality of slabs in the form of cross-sectioned slices, by dividing an entire three-dimensional CT image into the slabs each of which is approximately 1 cm thick, for example. The image processing function 443 is configured to generate projection images obtained by projecting each of the slabs in the thickness direction.

For example, when performing an average value projection, the converting function 445 is configured to calculate a color code of each of the pixels in the projection images, by calculating an average color value in the thickness direction of the slabs.

In another example, when performing a maximum value projection, the converting function 445 is configured to calculate a color code of each of the pixels in the projection images, by calculating a maximum value in the thickness direction of the slabs among brightness levels or opacity levels and further integrating, in the thickness direction, average hue values and the maximum brightness values.

The display controlling function 446 is configured to cause the display 42 to display a display screen including the plurality of projection images generated with respect to the slabs. On the display screen, the plurality of projection images are displayed in a list view on the single screen, similarly to the multiple cross-section display of the color superimposed images illustrated in FIG. 5, for example.

In the above embodiments, the example was explained in which the color image corresponding to the categories of the “local uniformity dominance” and the “contrast enhancement dominance” is generated; however, possible embodiments are not limited to this example. For instance, the colors in the color image may be assigned to organs of which the regions have been extracted by segmentation or may be assigned to a result of a perfusion analysis (e.g., Myocardial Blood Flow, MBF or Coronary Flow Reserve, CFR) performed on a myocardial region. The elements to which the colors are assigned in the color image are examples of the first state and the second state.

In the above embodiments, the color conversion mathematical functions are defined in such a manner that the display modes of the colors exhibited according to the color codes continuously change in response to the changes in the pixel values, while the hue assigned to the first state is not duplicate of the hue assigned to the second state in a major part thereof. In this situation, when the hue assigned to the first state is not duplicate of the hue assigned to the second state in the major part thereof, it means that in a connection part between the first state and the second state, the hues may be continuous (e.g., black may be used due to the brightness values being small), but in the regions where one of the first and the second states is stronger (e.g., the regions positioned distant from the connection part), the hues corresponding to the different states are different from each other. Further, in the above embodiments, the color conversion mathematical functions are defined in such a manner that the display modes such as the colors continuously change, as a whole, in accordance with the pixel values, but there are certain partial regions where the display modes such as the colors do not change in accordance with the pixel values. In other words, by using the color conversion mathematical functions defined as described above, the converting function 445 is configured to generate the display modes that include: the regions in which the display modes continuously change in accordance with the spatial or temporal changes in the data values of the pixels in the contrast-enhanced images corresponding to the plurality of temporal phases; and the regions in which the display modes do not change in accordance with the spatial or temporal changes.

The term “processor” used in the above explanations denotes, for example, a Central Processing Unit (CPU), a Graphics Processing Unit (GPU), or a circuit such as an ASIC or a Programmable Logic Device (PLD). Examples of the PLD include Simple Programmable Logic Devices (SPLDs), Complex Programmable Logic Device (CPLDs), and Field Programmable Gate Arrays (FPGAs). The one or more processors are configured to realize the functions by reading and executing the programs saved in a storage circuit. The storage circuit having the programs saved therein is a non-transitory computer-readable recording medium. Further, instead of having the programs saved in the storage circuit, it is also acceptable to directly incorporate the programs into the circuitry of the one or more processors. In that situation, the one or more processors are configured to realize the functions by reading and executing the programs incorporated in the circuitry thereof. Further, instead of executing the programs, it is also acceptable to realize the functions corresponding to the programs, by using a combination of logical circuits. Further, the processors of the present embodiments do not each necessarily have to be structured as a single circuit. It is also acceptable to structure one processor by combining together a plurality of independent circuits so as to realize the functions thereof. Furthermore, it is also acceptable to integrate two or more of the constituent elements illustrated in FIG. 1 in one processor so as to realize the functions thereof.

According to at least one aspect of the embodiments described above, it is possible to generate the display modes that contribute to increasing the speed of the medical image diagnosing processes in the contrast-enhanced dynamic examination.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

In relation to the embodiments described above, as certain aspects and selective characteristics of the present disclosure, the following notes are provided:

### Note 1:

A medical image processing apparatus including processing circuitry configured:
to obtain contrast-enhanced images related to an examined subject and corresponding to a plurality of temporal phases;
to determine, on the basis of data values of pixels in the contrast-enhanced images corresponding to the plurality of temporal phases, which one of contrast enhancement dominance and fluid accumulation dominance corresponds to the pixels or a region including the pixels; and
to generate a display mode based on the determination.

### Note 2:

The processing circuitry may be configured to determine which one of the contrast enhancement dominance and the fluid accumulation dominance corresponds to the pixels or the region including the pixels, on the basis of temporal changes in the data values of the pixels in the contrast-enhanced images corresponding to the plurality of temporal phases.

### Note 3:

With respect to each of the pixels or each of regions including the pixels, the processing circuitry may be configured to generate a color image in which color codes have been determined by using color conversion mathematical functions each of which individually corresponds to a different one of the contrast enhancement dominance and the fluid accumulation dominance.

### Note 4:

The color conversion mathematical functions may be configured to convert, into the color codes, variable values related to spatial or temporal changes in the data values of the pixels in the contrast-enhanced images corresponding to the plurality of temporal phases.

### Note 5:

The color conversion mathematical function corresponding to the contrast enhancement dominance and the color conversion mathematical function corresponding to the fluid accumulation dominance may be configured to convert a value of at least one of the variable values into a mutually same color code.

### Note 6:

The processing circuitry may further be configured to cause a display to display a display screen including one selected from between: a color superimposed image obtained by superimposing the color image on an image based on the contrast-enhanced images corresponding to the plurality of temporal phases; and the color image.

### Note 7:

The display screen may further include the contrast-enhanced images corresponding to the plurality of temporal phases.

### Note 8:

The display screen may include one selected from between the color superimposed image and the color image in each of a plurality of slice positions.

### Note 9:

A medical image processing apparatus including processing circuitry configured:
to obtain contrast-enhanced images related to an examined subject and corresponding to a plurality of temporal phases;
to calculate, on the basis of data values of pixels in the contrast-enhanced images corresponding to the plurality of temporal phases, a first color code corresponding to contrast enhancement dominance and a second color code corresponding to fluid accumulation dominance with respect to each of the pixels or each of regions including the pixels; and
to generate a color image in which color codes of pixels have been determined on the basis of the first color code and the second color code.

### Note 10:

A medical image processing apparatus including processing circuitry configured:
to obtain contrast-enhanced images related to an examined subject and corresponding to a plurality of temporal phases;
to determine, on the basis of data values of pixels in the contrast-enhanced images corresponding to the plurality of temporal phases, which one of a first state and a second state being different from the first state corresponds to the pixels or a region including the pixels; and
to generate a display mode based on the determination.

### Note 11:

The processing circuitry may be configured to generate the display mode that includes: a region in which the display mode continuously changes in accordance with spatial or temporal changes in the data values of the pixels in the contrast-enhanced images corresponding to the plurality of temporal phases; and a region in which the display mode does not change in accordance with the spatial or temporal changes.

### Note 12:

The processing circuitry may be configured to generate the display mode in such a manner that, with respect to the pixels or a first region including the pixels being in the first state and the pixels or a second region including the pixels being in a second state, mutually-different hues are assigned to regions each positioned distant from a connection part between the first region and the second region.

### Note 13:

A medical image processing method including:
obtaining contrast-enhanced images related to an examined subject and corresponding to a plurality of temporal phases;
determining, on the basis of data values of pixels in the contrast-enhanced images corresponding to the plurality of temporal phases, which one of contrast enhancement dominance and fluid accumulation dominance corresponds to the pixels or a region including the pixels; and
generating a display mode based on the determination.

### Note 14:

A computer program product storing a program executed by a computer, the program causing the computer to execute:
obtaining contrast-enhanced images related to an examined subject and corresponding to a plurality of temporal phases;
determining, on the basis of data values of pixels in the contrast-enhanced images corresponding to the plurality of temporal phases, which one of contrast enhancement dominance and fluid accumulation dominance corresponds to the pixels or a region including the pixels; and
generating a display mode based on the determination.

What is claimed is:

1. A medical image processing apparatus comprising processing circuitry configured:
to obtain contrast-enhanced images related to an examined subject and corresponding to a plurality of temporal phases;
to determine, on a basis of data values of pixels in the contrast-enhanced images corresponding to the plurality of temporal phases, which one of contrast enhancement dominance and fluid accumulation dominance corresponds to the pixels or a region including the pixels; and
to generate a display mode based on the determination.

2. The medical image processing apparatus according to claim 1, wherein the processing circuitry is configured to determine which one of the contrast enhancement dominance and the fluid accumulation dominance corresponds to the pixels or the region including the pixels, on a basis of temporal changes in the data values of the pixels in the contrast-enhanced images corresponding to the plurality of temporal phases.

3. The medical image processing apparatus according to claim 1, wherein, with respect to each of the pixels or each of regions including the pixels, the processing circuitry is configured to generate a color image in which color codes have been determined by using color conversion mathematical functions each of which individually corresponds to a different one of the contrast enhancement dominance and the fluid accumulation dominance.

4. The medical image processing apparatus according to claim 2, wherein, with respect to each of the pixels or each of regions including the pixels, the processing circuitry is configured to generate a color image in which color codes have been determined by using color conversion mathematical functions each of which individually corresponds to a different one of the contrast enhancement dominance and the fluid accumulation dominance.

5. The medical image processing apparatus according to claim 3, wherein the color conversion mathematical functions are configured to convert, into the color codes, variable values related to spatial or temporal changes in the data values of the pixels in the contrast-enhanced images corresponding to the plurality of temporal phases.

6. The medical image processing apparatus according to claim 4, wherein the color conversion mathematical functions are configured to convert, into the color codes, variable values related to spatial or temporal changes in the data values of the pixels in the contrast-enhanced images corresponding to the plurality of temporal phases.

7. The medical image processing apparatus according to claim 5, wherein the color conversion mathematical function corresponding to the contrast enhancement dominance and the color conversion mathematical function corresponding to the fluid accumulation dominance are configured to convert a value of at least one of the variable values into a mutually same color code.

8. The medical image processing apparatus according to claim 6, wherein the color conversion mathematical function corresponding to the contrast enhancement dominance and the color conversion mathematical function corresponding to the fluid accumulation dominance are configured to convert a value of at least one of the variable values into a mutually same color code.

9. The medical image processing apparatus according to claim 3, wherein the processing circuitry is further configured to cause a display to display a display screen including one selected from between: a color superimposed image obtained by superimposing the color image on an image based on the contrast-enhanced images corresponding to the plurality of temporal phases; and the color image.

10. The medical image processing apparatus according to claim 4, wherein the processing circuitry is further configured to cause a display to display a display screen including one selected from between: a color superimposed image obtained by superimposing the color image on an image based on the contrast-enhanced images corresponding to the plurality of temporal phases; and the color image.

11. The medical image processing apparatus according to claim 9, wherein the display screen includes the contrast-enhanced images corresponding to the plurality of temporal phases, as well as one selected from between: the color superimposed image in each of a plurality of slice positions; and the color image in each of the plurality of slice positions.

12. The medical image processing apparatus according to claim 10, wherein the display screen includes the contrast-enhanced images corresponding to the plurality of temporal phases, as well as one selected from between: the color superimposed image in each of a plurality of slice positions; and the color image in each of the plurality of slice positions.

13. A medical image processing apparatus comprising processing circuitry configured:
to obtain contrast-enhanced images related to an examined subject and corresponding to a plurality of temporal phases;
to calculate, on a basis of data values of pixels in the contrast-enhanced images corresponding to the plurality of temporal phases, a first color code corresponding to contrast enhancement dominance and a second color code corresponding to fluid accumulation dominance with respect to each of the pixels or each of regions including the pixels; and
to generate a color image in which color codes of pixels have been determined on a basis of the first color code and the second color code.

14. A medical image processing apparatus comprising processing circuitry configured:
to obtain contrast-enhanced images related to an examined subject and corresponding to a plurality of temporal phases;
to determine, on a basis of data values of pixels in the contrast-enhanced images corresponding to the plurality of temporal phases, which one of a first state and a second state being different from the first state corresponds to the pixels or a region including the pixels; and
to generate a display mode based on the determination.

15. The medical image processing apparatus according to claim 14, wherein the processing circuitry is configured to generate the display mode that includes: a region in which the display mode continuously changes in accordance with spatial or temporal changes in the data values of the pixels in the contrast-enhanced images corresponding to the plurality of temporal phases; and a region in which the display mode does not change in accordance with the spatial or temporal changes.

16. The medical image processing apparatus according to claim 14, wherein the processing circuitry is configured to generate the display mode in such a manner that, with respect to the pixels or a first region including the pixels being in the first state and the pixels or a second region including the pixels being in a second state, mutually-different hues are assigned to regions each positioned distant from a connection part between the first region and the second region.

17. The medical image processing apparatus according to claim 15, wherein the processing circuitry is configured to generate the display mode in such a manner that, with respect to the pixels or a first region including the pixels being in the first state and the pixels or a second region including the pixels being in a second state, mutually-different hues are assigned to regions each positioned distant from a connection part between the first region and the second region.

18. A medical image processing method comprising:
obtaining contrast-enhanced images related to an examined subject and corresponding to a plurality of temporal phases;
determining, on a basis of data values of pixels in the contrast-enhanced images corresponding to the plurality of temporal phases, which one of contrast enhancement dominance and fluid accumulation dominance corresponds to the pixels or a region including the pixels; and
generating a display mode based on the determination.

19. A computer program product storing a program executed by a computer, the program causing the computer to execute:
obtaining contrast-enhanced images related to an examined subject and corresponding to a plurality of temporal phases;
determining, on a basis of data values of pixels in the contrast-enhanced images corresponding to the plurality of temporal phases, which one of contrast enhancement dominance and fluid accumulation dominance corresponds to the pixels or a region including the pixels; and
generating a display mode based on the determination.
